(19) United States
(12) Patent Application Publication
(10) Pub. No.: US 2013/0312192 A1
(43) Pub. Date: Nov. 28, 2013

(54) Pillow Device

(71) Applicant: Munsoo LEE, Los Angeles, CA (US)

(72) Inventor: Munsoo LEE, Los Angeles, CA (US)

(21) Appl. No.: 13/480,338

(22) Filed: May 24, 2012

(51) Int. Cl. A47G 9/10; A47C 31/00; A61F 9/04

(52) U.S. Cl. 5/639

(57) ABSTRACT

A pillow device having a pillow portion with a main head support section for supporting the head, and at least one eye mask portion for covering the eyes of the user. For additional comfort, a neck support may be removably attached to the pillow device to provide not only support for the head, but also support for the neck. Because of the overall shape of the pillow device can easily simulate a doll, a skirt with popping out figures may be added to simulate an angel or other favorite figures with open arms to welcome the user. Moreover, the pillow device may function as a computer pad holder and a bag.

### BACKGROUND OF THE INVENTION

The present invention relates to a pillow device with one or more eye mask covers for supporting a head of the user so that the pillow device would promote sleep or rest. Although it is a common practice to have a pillow or a neck supporting pillow and an eye mask, each used separately, there has been no device that combined an eye mask with a pillow, and there has been no device that combined an eye mask with a pillow and a neck support. Therefore, especially during an air flight, passenger had to bring his or her own neck support (common “U” shaped pillow) and bring either or both a separate pillow to be comfortable (the pillows airlines provide are often too small and too skimpy to be comfortable) and an eye mask. Although some international flights provided eye masks and pillows, the passengers could not always count on the airlines, especially these days in which the airlines are trying to cut cost, to provide sufficient pillows and eye masks.

Accordingly, a need for a combined pillow device that integrates a pillow, a neck support, and an eye mask into a single device so the passengers can have a comfortable head rest, a comfortable neck support, and a comfortable eye mask to help them sleep and to rest whether in transit (in any vehicle) or in their homes (whether sitting on favorite chairs, sofas, or lying down on their beds). Accordingly, this invention is directed to solve these problems and satisfy a very long-felt need.

### SUMMARY OF THE INVENTION

The present invention contrives to solve the disadvantages of having each of the three items separately when traveling or resting: a pillow, a neck support, and an eye mask. One thing amazes the inventor is that, for more than one hundred years of air flight and thousands of years of vehicle travel, no one had combined these three items into a single device. Another greater mystery is that no one had combined these three into a single device for medical care, especially for patients who need comfortable pillows and substantial neck support such that they may peacefully sleep or rest better with their eyes covered with a soft eye mask. Therefore, an object of the invention is to provide the most comfortable sleep or rest during air flight or as a person tries to rest or sleep on a chair, a couch, a sofa, or a bed.

According to this invention, a pillow device has a pillow portion with a main head support section for supporting the head, and at least one eye mask portion for covering the eyes of the user. The eye mask portion is attached, or alternately removably attached, to the pillow portion. The length of the eye mask portion may be adjusted when the detachably attaching means is used. The eye mask portion has a section designated to cover one or more eyes so the user may block out the light. A possible added advantage is that because the eye mask portion wraps around the head, when it covers an eye, it also covers an ear to block out some noise without inserting a noise reducing foam into an ear to help the person rest better.

The eye mask portion has two ends: a first end attached to the pillow portion and a second end. Substantially distant from the first end is the eye covering section, such that the eye covering section would comfortably cover one eye. As an alternate design, when the eye mask portion is made extra long, then a larger eye covering section may cover both eyes of the user and the second end (the distal end from the first end) may detachably attach to the pillow portion.

The fastening devices used to detachably attach either the first end or the second end of the eye mask portion may be one of varieties of fastening devices available. One example may be a hook and pile fastening device, snap on tacks, magnets, buttons & eyelets, and even tie straps. The inventor believes a use of hook and pile fastening device works the best, but any available means may be used. These removably attaching devices, the fastening devices, should be located near the second end. However, for convenience of use to fit many different sized heads and also for varying the tightness of the eye mask portion around the head, it is preferable that fastening means, such as hook and pile straps, may run along a substantial length of the eye mask portion.

A pillow cushion inserted in the pillow portion may be of any material. Some people may prefer soft foam (such as memory foam), air inflatable bag, or down fill, but it is foreseeable to use substantially hard materials, such as beans, grains, a roll of bamboo strips, or a soft wooden block, to better suit some users of certain Asian culture.

As a supplemental part to the pillow device, a neck support, substantially formed in a “U” shape to support and comfortably hold the neck of the user within the inner portion of the “U” shape, may be attached to the pillow portion. The pillow portion and the neck support may be detachably joined together or fixedly joined together at about an adjoining pillow side (a side of the pillow portion) and an adjoining neck support side (a side of the neck support).

One version of the pillow device may have both the adjoining pillow side and the adjoining neck support side having sloped surfaces such that when the pillow portion and the neck support are joined together, two attached parts would form an angle. Pending the angle of each slope, these two parts can be joined together to form any varying angles. An example may be that both surfaces having about 50° would make the joined angle to be about 100°, and both surfaces having about 45° would make the joined angle to be about 90° (about perpendicular) or about 180° (about flat or horizontal) when either the pillow portion or the neck support is turned around. The angles may be varied, such that one side may have 30° and the other side may have 60° or 70° to better fit the head and the neck of the user in a most restful position while sitting.

One advantage of the pillow portion and the neck support detachably attached or joined together lengthwise on an adjoining edge is that the neck support may be detached from the pillow portion and its upper portion may be laid flat against the head resting portion of the pillow portion to be packaged compactly against the neck support. A preferred method of joining together the pillow portion and the neck support is using a lengthwise zipper or a plurality of magnets or buttons. In this configuration, because the eye mask portions can actually wrap around both the pillow portion and the neck support, the pieces will be held securely together. On the opposite side of the adjoining edge may have a securing strap attached to either the pillow portion or the neck support to help secure the respective position of the pillow portion and the neck support substantially at either about 90 degree or about 180 degree position when the pieces are joined together.

Because the pillow device has a large head rest area and the neck support is shaped in a substantially “U” shape, a various doll designs may be formed when the neck support is attached to the pillow portion. Therefore, in order to make the pillow device simulate a doll better, the neck support may further comprise of a skirt that may be rolled up or rolled down to simulate arms, a tail, or a dress. The skirt may be secured or removably attached on the neck support, and either or both the inside and the outside of the skirt may have various designs and patterns on them to depict various images.

Also, the pillow device may have an additional strap that can be inserted into the pillow portion or into the neck support and taken out to be a handle for easy carry or to be a tie-down belt around a back of the chair. In addition, a musical device may be inserted into the pillow device to enhance the sleep or the rest the user seeks. This musical device may be a music box (for example, a toy with a pull handle that makes a recorded sound when pulled). MP3 player, a phone, or a speaker that can be plugged into an outside source (such as an airplane's seat speaker output plug).

For better accommodating the cold ambient temperature in many airplanes, the pillow device may have an internal (or external) heater or cooler attached to one or more of the pillow portion, eye mask portion, or neck support. Also, a projector may be inserted in one or more of the neck support ends to project video onto a ceiling or onto a screen. Moreover, an aroma source may be inserted in the pillow device to help the person to relax.

Although the present invention is briefly summarized, the fuller understanding of the invention can be obtained by the following drawings, detailed description and appended claims.

### BRIEF DESCRIPTION OF THE DRAWINGS

These and other features, aspects and advantages of the present invention will become better understood with reference to the accompanying drawings, wherein:

FIG. 1 is a perspective view showing an embodiment according to the invention.

FIG. 2 is a perspective view showing another embodiment according to the invention.

FIG. 3 is various perspective views showing the embodiment shown in FIG. 2.

FIG. 4 is various perspective views showing the embodiment shown in FIG. 2 in various configurations.

FIG. 5 is various perspective views showing an improved embodiment of the invention shown in FIG. 2 in various configurations.

FIG. 6 is various side views showing different uses of the invention.

FIG. 7 is various perspective views showing another embodiment of the invention.

FIG. 8 is various perspective views showing yet another embodiment of the invention.

FIG. 9 is various perspective views showing yet another embodiment of the invention.

FIG. 10 is various perspective views showing yet another embodiment of the invention.

FIG. 11 is a perspective views showing yet another embodiment of the invention.

FIG. 12 is a perspective views showing yet another embodiment of the invention.

FIG. 13 is a perspective views showing yet another embodiment of the invention.

FIG. 14 is a perspective views showing yet another embodiment of the invention.

FIG. 15 is a perspective views showing yet another embodiment of the invention.

FIG. 16 is a perspective views showing yet another embodiment of the invention.

### DETAILED DESCRIPTION EMBODIMENTS OF THE INVENTION

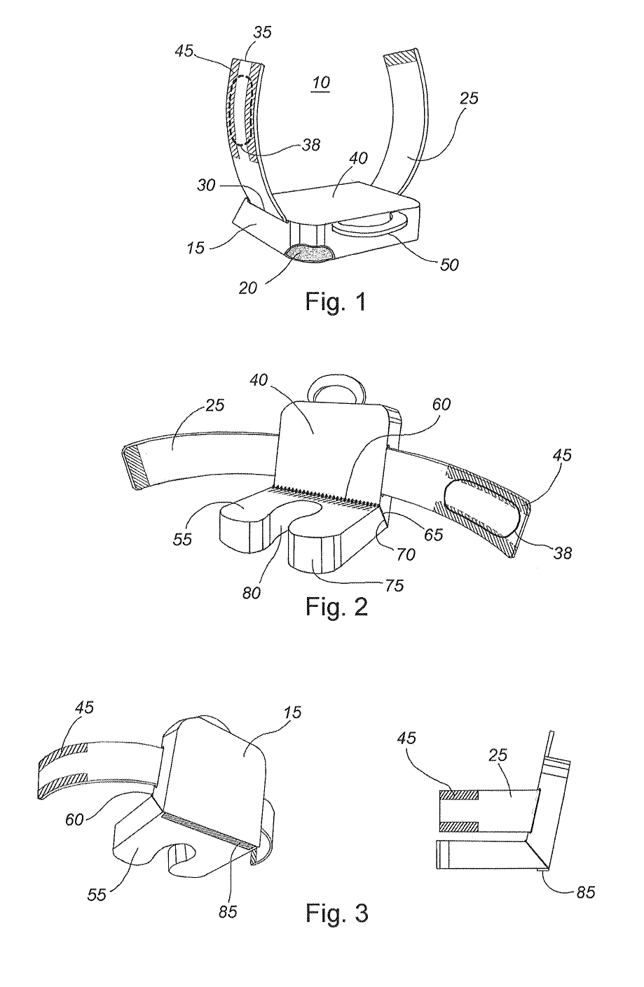

Referring to the figs, FIG. 1 shows a perspective view of a pillow device 10 according to the invention. The pillow device 10 has a pillow portion 15 with a pillow cushion 20 inside. On each side of the pillow portion 15 has an eye mask portion 25. Each eye mask portion 25 has a first end 30 and a second end 35, wherein the first end 30 is attached (or detachably attached) to the pillow portion 15 and the second end 35 at the distal end from the first end 30. The eye mask portion 25 has an eye covering section 38 on the inside portion of the eye mask portion 25, and the eye covering section 38 may be a small or large area inside of the eye mask portion 25 to better accommodate the preference of a user. The eye covering section 38 should be made of soft material, such as soft terrycloth or smooth fabric.

On top of the pillow portion 15 is a head support 40 where the head of user would comfortably rest. With two eye mask portions 25 wrapping around the head, the user is comfortably embraced by the pillow device, with the eye mask potions 25 covering the eyes to block out the light and to comfort around the eyes and the head comfortably resting upon the head support 40 simulating the user's favorite pillow. Because the fastening devices 45 are attached on the eye mask portions 25, the eye mask portions 25 can actually not only securely hold the second ends 35 of the eye mask portions 25 together, but also apply adjustable pressure on and around the eyes. The fastening devices 45 may be a hook and pile fastening device, magnets, plurality of spaced apart snap on tacks, plurality of spaced apart buttons & eyelets, and even tie straps. The inventor recommends that the selection of the fastening devices should be done with care to prevent accidental choking during the sleep.

Although FIG. 1 shows the pillow device 10 having two eye mask portions 25 attached to the pillow portion 15, it is possible to have the eye mask portions 25 detachably attached to the pillow portion 15. In fact, when they are detached from the pillow portion 15, they can be inserted into a pocket (not shown) on the pillow portion 15 or even into the pillow portion 15 for easy carry. Moreover, a handle 50 may be attached to a side of the pillow portion 15 to help carry the pillow device 10. Furthermore, although FIG. 1 shows the pillow device 10 having two eye mask portions 25 attached to the pillow portion 15, it is possible to have only one eye mask portion 25 (designed longer) detachably attached to the pillow portion 15 at the first end 30, eye mask portion 25 wrap around the entire head, and the second end 35 detachably attached to the pillow portion 15.

FIG. 2 shows an embodiment of the invention with a neck support 55 detachably attached to the pillow portion 15 at an adjoining edge 60 using a zipper or a lengthwise hook and pile fastening device 45. At the adjoining edge 60, an adjoining pillow side 65 of the pillow portion 15 and an adjoining neck support side 70 of the neck support 55 come together to form an angle between the head support 40 and an upper surface of the neck support 55. As the slope on the adjoining pillow side 65 and the slope on the adjoining neck support side 70 vary, the angle between the head support 40 and an upper surface of the neck support 55 will vary. In FIG. 2, the slopes are slightly more than 45° each, so that the overall angle between the head support 40 and an upper surface of the neck support 55 is somewhat larger than 90°.

The neck support 55 is generally in a “U” shape, so it has two neck support ends 75 away from the adjoining edge 60, and an inner portion 80 of the “U” shape in between two neck support ends 75. With the neck support 55, the user can sit on a chair (as in an airliner) and have the head resting upon the head support 40, the eyes covered with eye mask portions 25, and the neck embraced in a secure and comfortable position in the inner portion 80 of the “U” shape to optimize the comfort for resting.

FIG. 3 shows the pillow portion 15 and the neck support 55 held securely by a securing strap 85 at the underside of the pillow device. Because the pillow portion 15 and the neck support 55 are joined together at the adjoining edge 60, the securing strap 85 at the bottom can more fixedly hold the pillow portion 15 and the neck support 55 together, better ensuring that the pillow portion 15 and the neck support 55 do not wiggle/shift about to an awkward position during the rest. Optionally, either or both the pillow portion 15 and the neck support 55 may have contoured surfaces to better fit the user's head, neck, and shoulder. The contours when used should be mirror images so that the pillow portion 15 and the neck support 55 would fit without a gap when put side by side upon folding.

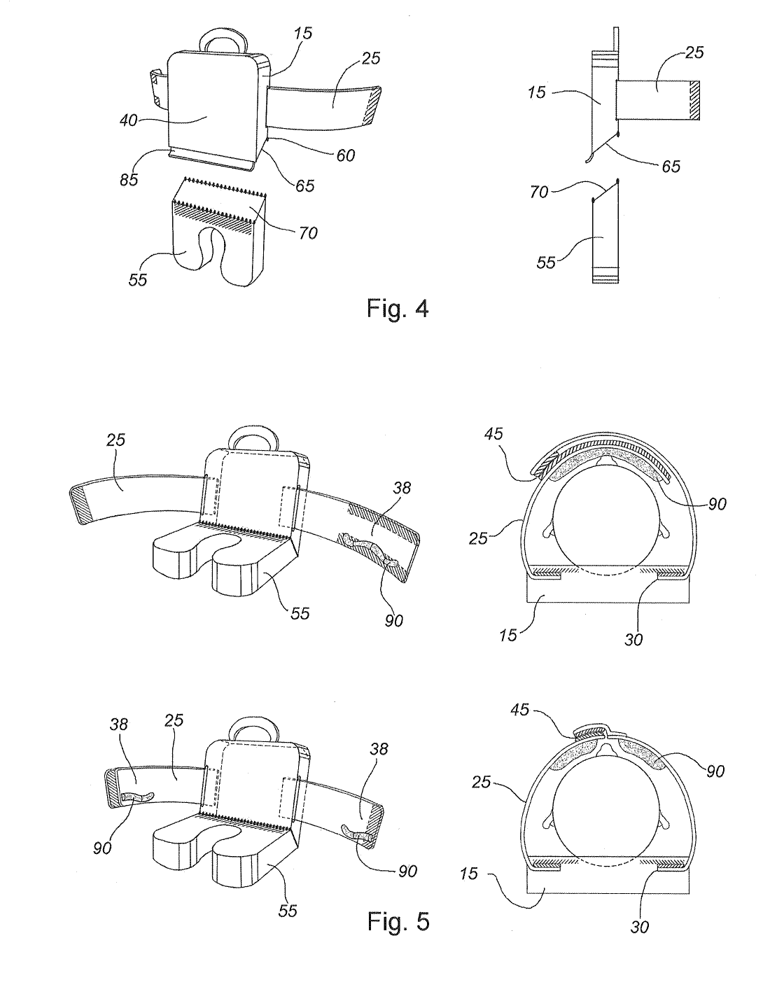

FIG. 4 shows the pillow portion 15 and the neck support 55 detached, with the pillow portion 15 above the neck portion. Compared to the FIG. 2 and FIG. 3, the pillow portion 15 is turned around to show that when the slope on the adjoining pillow side 65 and the slope on the adjoining neck support side 70 are both about 45°, simply reversing either the pillow portion 15 or the neck support 55 will make the overall angle between the head support 40 and an upper surface of the neck support 55 to be 180° (flat). FIG. 4 also shows a second adjoining edge 60 having a second set of fastening device to fasten with the existing fastening device at the adjoining edge 60.

FIG. 5 shows the eye mask portions 25 removably attached to the pillow portion 15; in fact, removably attached inside the pillow portion 15. This configuration allows the eye mask portions 25 to be completely removed and stored away (perhaps in the pillow device 10) when not in use. When the first end 30 of the eye mask portion 25 is made insertable into the pillow portion 15, then the length of the eye mask portion 25 may be adjustable. Also, the eye covering section has an eye padding 90 to block additional light that may pass through because of the height of the nose and, if the user so desires, to avoid the eye covering section from pressing down on the eyes. The upper FIG. 5 shows one eye mask portion 25 covering both eyes, and the lower FIG. 5 shows two eye mask portions 25, each covering one eye.

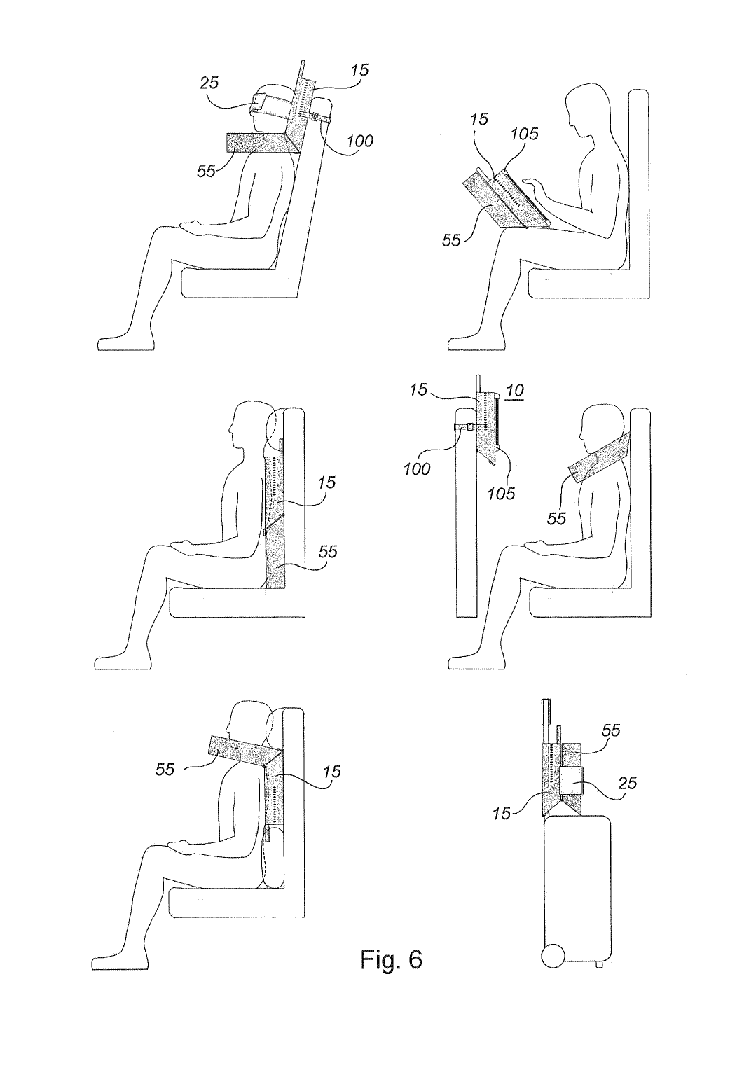

FIG. 6 shows various uses of the pillow device. The pillow device 10 may have an additional strap 100 (either of a fixed length or an expandable or stretchable strap) to wrap around the back of a chair. The pillow device 10 may have a computer pad tabs 105 that securely holds a computer pad (for example a tablet PC) or laptop computer while the pillow device 10 is resting on the knees of the user. Because the pillow device 10, both the pillow portion 15 and the neck support 55, is mostly made of soft and cuddly material, the pillow device 10 can be positioned lengthwise to function as a back support. Moreover, because the pillow portion 15 and the neck support 55 may be detached, the pillow portion 15 with a computer pad can securely held by a chair front of the user while the user enjoys the video on the computer pad, while his neck is firmly secured by the neck support 55. Furthermore, the pillow portion 15 and the neck support 55 may be configured to function as a neck support 55 and a back support. Furthermore, the pillow device 10 may be modified to have an extra strap forming a through hole (an opening) to pass a luggage handle through for easy transportation on a pull-out handle luggage through the airports.

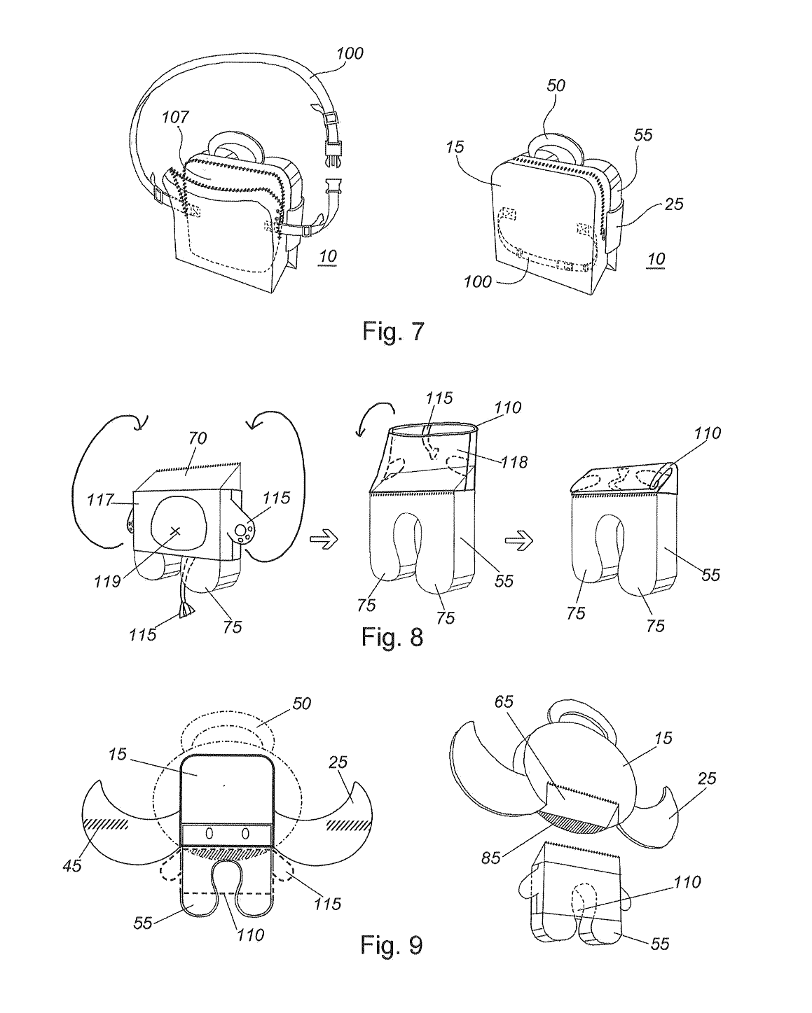

FIG. 7 shows yet another use of the pillow device 10. The pillow device 10 has a bag portion 107 with an opening at the rear of the pillow portion 15 in which the additional strap 100 can be inserted as well as carry additional items. The opening of the bag is shown with a common zipper as shown in this Fig.

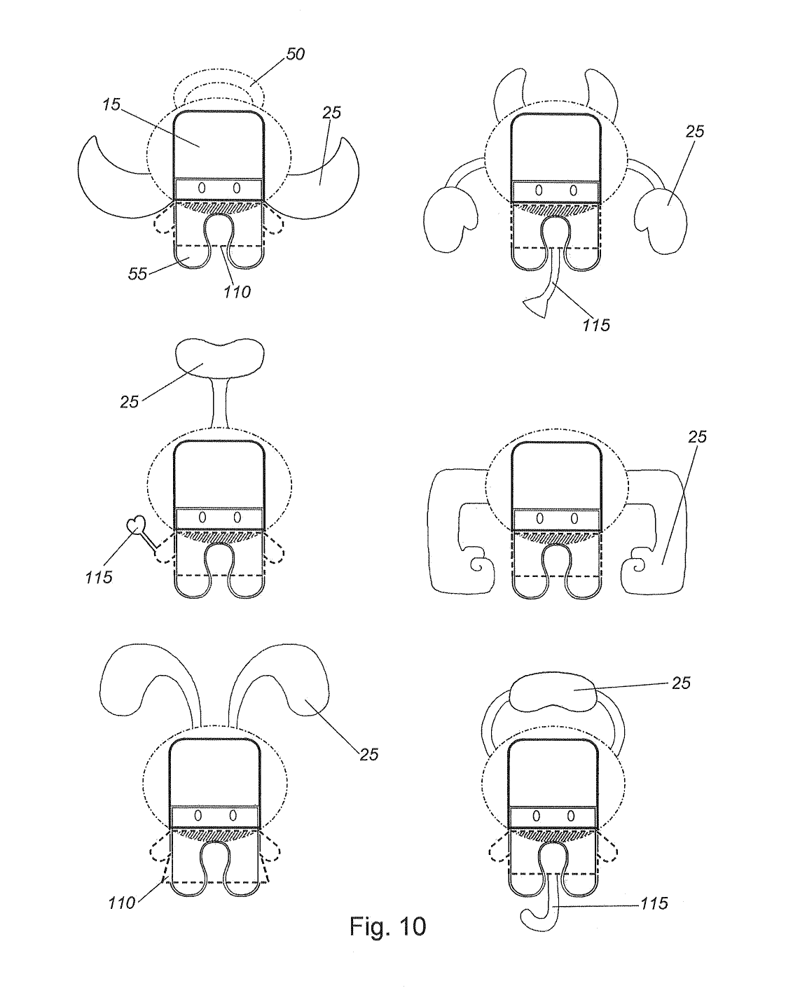

FIGS. 8-10 show a skirt 110 with various figures 115 attached to it; thus attached to the neck support 55. Either or both the inside 118 and the outside 117 of the skirt 110 may have various decorations 119 and patterns on them to depict various images. The skirt 110 may be rolled down or rolled up and folded to be stored away in between the adjoining pillow side 65 and the adjoining neck support side 70.

The inside 118 of the skirt 110 may have various figures 115 (such as arms, a tail, a flag, a toy gun, etc. . . . ) flexibly attached to the inside 118 of the skirt 110, so that when the skirt 110 is rolled down to display the inside decoration, the figures 115 would either open up or pop up to greet the user.

Therefore, as an example, the pillow device 10 can simulate a child or an angel or an animal with a face (on the pillow portion 15; for example, a print of a face of an angel wearing a tiara), the skirt 110 rolled down to reveal two arms and hands with a wand, and two neck support ends 75 depicting the legs. A novelty can be easily seen when the skirt rolls down, the arms hidden in the skirt 110 can pop out as two open arms, instantly greeting the user. If an angel is depicted, various design and decorations may be printed on the pillow portion 15 to simulate the angel's face, rolled down skirt may reveal two open arms with a wand in one hand and a pair of wings on the back side, and the two distal ends of the neck support end 75 simulate two legs capped with two shoes (not shown).

Also as shown in the FIGS. 8-10, the eye mask portions 25 may be shaped in various means, including and not limited to a pair of wings, a pair of hands, a chef's hat, an alien antenna, a pair of bunny ears, a tiara, and various other figures. The eye mask portions 25 as such may be designed to suit a young user.

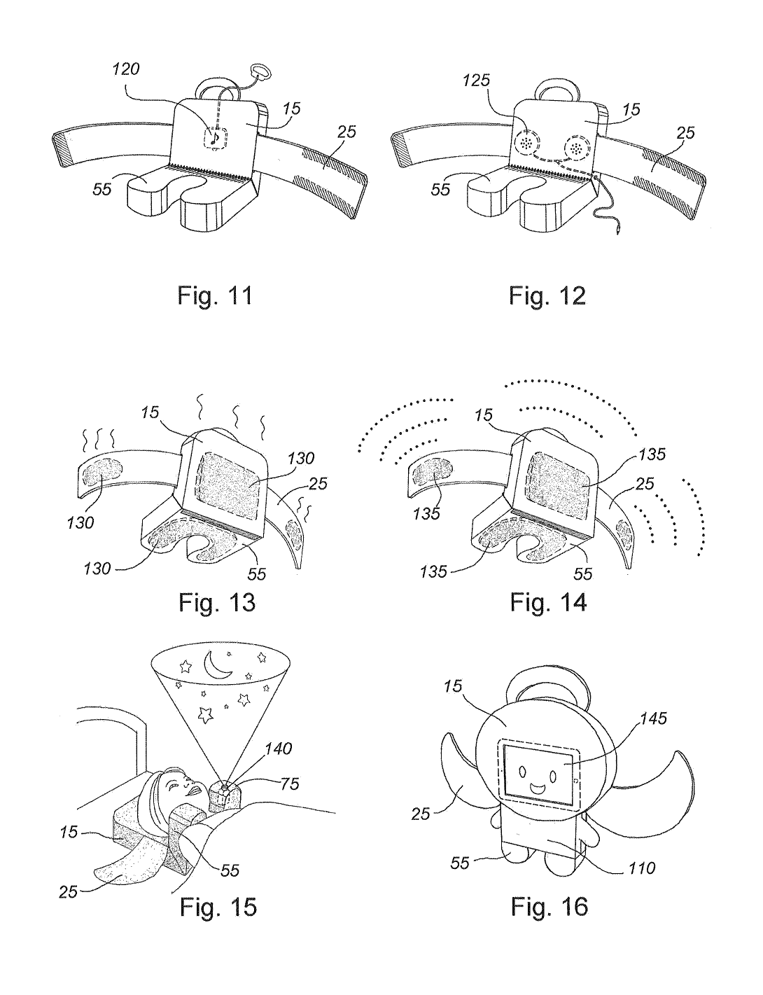

FIGS. 11-12 show the pillow portion 15 having a musical device 120 inserted in it so the user may listen to her favorite sounds while resting or seeking sleep. This musical device 120 may be a music box. MP3 player, a phone, or a speaker 125 that can be plugged into an outside source (such as an airplane's seat speaker output plug).

FIG. 13 shows the pillow portion 15 and the eye mask portions 25 having heaters 130 to help against the cold ambient temperature in many airplanes. Also, the neck support 55 may also have a heater 130 to warm around the neck. Alternately, the heaters 130 may be replaced by coolers.

FIG. 14 shows the pillow portion 15 and the eye mask portions 25 having aroma source 135 to help against the cold ambient temperature in many airplanes. Also, the neck support 55 may also have an aroma source 135 to help the user to relax or to sleep. Alternately, the aroma sources may be used with combination of heaters 130 or coolers.

FIG. 15 shows the neck support 55 having a projector 140 located in one or more of the neck support end 75 to project video onto a ceiling or onto a screen. This embodiment may be particularly good for the users who need to count sheep or watch T.V. in order to fall asleep better. A warmly heated and cuddly pillow device 10 that plays favorite calming music with a video showing calming pictures may invite even a person suffering from insomnia to readily fall asleep. Alternately, a warmly heated and cuddly pillow device 10 that plays favorite calming music with the eyes warmly wrapped around with soft eye mask portions 25 may also easily invite even a person who has difficulties to sleep in flight to readily fall asleep. The user of any heaters 130 in an enclosed space, such as under a blanket or in a pillow device 10 should be careful not to use temperatures that may cause burns; therefore, a temperature sensor to automatically turn off the heaters would be an improvement to this embodiment.

FIG. 16 shows the pillow device 10 having a monitor 145 or a computer pad placed in the pillow portion 15. The monitor 145 or computer pad can play a picture show of favorite photos or display a latest movie via wireless connection.

While the invention has been shown and described with reference to different embodiments thereof, it will be appreciated by those skilled in the art that variations in form, detail, compositions and operation may be made without departing from the spirit and scope of the invention as defined by the accompanying claims.

What is claimed is:

1. A pillow device comprising:
a pillow portion having a head support section for supporting a head of a user; and one or more eye mask portions 25 for covering eyes of the user attached to the pillow portion, wherein the eye mask portion 25 having an eye covering section.

2. The pillow device of claim 1, wherein the eye mask portion 25 having a first end attached to the pillow portion, and wherein the eye covering section is substantially distant from the first end so that the eye covering section is comfortably over one or more eyes of the user when the eye mask portion 25 wraps around the head of the user.

3. The pillow device of claim 2, wherein the eye mask portion 25 having a second end at the substantially opposite end from the first end of the eye mask portion 25, wherein a fastening device is attached at substantially near the second end, such that when two or more of the eye mask portions 25 are attached to the pillow portion, the fastening devices located near the second ends may be used to detachably attach the second ends.

4. The pillow device of claim 3, wherein the pillow device further comprises of a pillow cushion inserted within the pillow device to comfortably support the head of the user.

5. The pillow device of claim 3, wherein the pillow device further has an adjoining pillow side, and the pillow device further comprises of a neck support substantially formed in a “U” shape so that a neck of the user may be comfortable held within an inner portion of the “U” shape while the head resting on the pillow device, wherein the neck support further has an adjoining neck support side attached to the adjoining pillow side.

6. The pillow device of claim 5, wherein the neck support is removably attached to the pillow device.

7. The pillow device of claim 6, wherein the adjoining pillow side is sloped and the adjoining neck support side is also sloped.

8. The pillow device of claim 7, wherein the adjoining pillow side is sloped and the adjoining neck support side is also sloped so that when both the adjoining pillow side and the adjoining neck support side are joined together the angle formed between the head support section and the neck support section is about 90 degrees.

9. The pillow device of claim 8, wherein the adjoining pillow side is sloped at about 45 degrees and the adjoining neck support side is also sloped at about 45 degrees so that when both the adjoining pillow side and the adjoining neck support side are joined together, the angle formed between the head support section and the neck support section is about 90 degrees or the angle formed between the head support section and the neck support section is about 180 degrees when either the head support section or the neck support section is turned upside down.

10. The pillow device of claim 6, the neck support section may be held next to the head support section when the neck support section is detached from the head support section or bent over side by side at an adjoining edge, and one or more of the eye mask portion 25 may wrap around the neck support section to embrace the neck support section and hold the neck support section next to the head support section.

11. The pillow device of claim 10, wherein the pillow device has a securing strap attached to either the pillow portion or the neck support to help secure the respective position of the pillow portion and the neck support at either about 90 degree or about 180 degree position.

12. The pillow device of claim 5, wherein the neck support further comprises of a skirt having a secured end, an open end, an outside, and an inside, wherein the skirt substantially wraps around the neck support, and wherein the secured end may be removably attached.

13. The pillow device of claim 12, wherein the open end of the skirt may be rolled out to display the inside of the skirt decorated with a decoration to depict various images.

14. The pillow device of claim 13, wherein the skirt further comprises of one or more figures flexibly attached to the inside of the skirt, so that when the skirt is rolled down to display the inside decoration, the figures would either open up or stand up to greet the user.

15. The pillow device of claim 14, wherein the flexible figures are substantially in a form of human or toy arms and hands, such that when the skirt is rolled down, the flexible figures would either open up or stand up as if greeting with the image's open arms.

16. The pillow device of claim 15, wherein the head support section is decorated with a face decoration so that when the skirt is rolled down, along with the flexible figures, the pillow device will depict a doll with the face decoration and flexible figures simulating a doll's body.

17. The pillow device of claim 3, wherein the pillow device further comprises a musical device to play music or a speaker device that receives data signal from an outside source.

18. The pillow device of claim 3, wherein the pillow device has an internal heater or a cooler.

18. The pillow device of claim 3, wherein the pillow device has an aroma source.

20. The pillow device of claim 3, wherein the neck support has a neck support end, wherein a projector is either imbedded in or attached to about the neck support end, such that a projector may project pictures or videos.
